(19) United States
(12) Patent Application Publication
(10) Pub. No.: US 2007/0275462 A1
(43) Pub. Date: Nov. 29, 2007

(54) Human Prostate Cancer Cell Factor(s) that Induce Stem Cell Commitment and Osteogenesis

(76) Inventors: Christopher L. Hall, Ypsilanti, MI (US); Michael Friedman, Philadelphia, PA (US); Michael W. Long, Northville, MI (US)

Correspondence Address: FULBRIGHT & JAWORSKI L.L.P., 600 CONGRESS AVE., SUITE 2400, AUSTIN, TX, 78701, US

(21) Appl. No.: 10/574,527

(22) PCT Filed: Oct. 1, 2004
(86) PCT No.: PCT/US04/32096

§ 371 Date: May 22, 2007

Related U.S. Application Data
(60) Provisional application No. 60/508,108, filed on Oct. 2, 2003.

(51) Int. Cl. C07K 14/51; C07K 14/00; C12N 5/06

(52) U.S. Cl. 435/377; 435/404; 530/350; 530/412

(57) ABSTRACT

Human prostate cancer (CaP) cells secrete biological activities that recruit cells to differentiate into the osteoblast lineage. Conditioned medium (CM) produced by metastatic human CaP cells (DuCaP and VCaP) induced commitment and differentiation of mesenchymal stem cells (MSC) into osteoblasts. CaP-CM induces cellular condensation into tissue-like aggregates. In turn, these tissue-like aggregates secrete and mineralize a bone matrix, forming bone outside the body (ex vivo). Thus, conditioned media and/or proteins isolated therefrom may be used to facilitate bone formation in fracture repair and bone diseases.

This application claims benefit of priority to U.S. Provisional Ser. No. 60/508,108, filed Oct. 2, 2003, the entire contents of which are hereby incorporated by reference.

The government owns rights in the present invention pursuant to grant number R01 DK061456.

### BACKGROUND OF THE INVENTION

1. Field of the Invention

The present invention relates generally to the fields of biology and medicine. More particularly, it concerns a process for the identification and isolation of factors from prostate cancer cells that induce the formation of bone and uses thereof.

2. Description of Related Art

Prostate cancer (CaP) is the second leading cause of cancer death in men with an estimated 189,000 men/year diagnosed as having prostate cancer. It is notable that 83% of all prostate cancers are discovered in local and regional stages. In 2002, an estimated 30,200 men are expected to die of prostate cancer.

A unique clinical feature of certain cancers is the formation of osteoblastic or bone-producing lesions in the pelvis and vertebral column, in which large amounts of bone are generated at the site of CaP metastasis. In fact, spine metastasis represents 90% of prostate cancer metastasis, and recurrence is common (45% risk within 2 years). However, despite significant research efforts, the molecular mechanism mediating this osteoblastic response as yet is unclear.

Current therapies consist of surgical intervention, radio-therapy, hormone therapy, and chemotherapy. All of these have extensive side effects, and are directed at eradication of the primary tumor, often missing metastatic lesions, or not preventing this process.

Conversely, the rate of bone fractures in the United States is estimated at 6,000,000 individuals per year. When a bone is completely fractured, a significant number of fractures require medical intervention beyond simple immobilization (casting), particularly those involving trauma A major problem in such instances is the lack of proximity of the two bone ends (referred to as non-union). This results in an inappropriate and prolonged repair process, which may prevent recovery. The average length of time for the body to repair a fracture is 25-100 days, for moderate load-bearing, and one year for complete repair. Thus, both simple fractures and medically complicated breaks would benefit from novel therapeutic modalities which accelerate and/or complete the repair process. The same is true for those bone diseases (referred to as osteopenias) which result in a thinning of the bone the primary symptom of which is an often-debilitating fracture, and other diseases in which bone strength or elasticity is compromised. While the bone formed as a result of osteoblastic Cap metastasis is increased in mass, it is improperly organized. Thus, this disorganized structure may weaken the bone itself resulting in increased risk of fracture.

A number of studies have evaluated the capacities of CaP cells or CaP cell conditioned media to affect osteogenesis. These studies have solely focused on the ability of CaP to directly influence osteoblast proliferation or development and are inconsistent. Thus, CaP Cm has been alternatively shown to both stimulate osteoblasts proliferation and development (LeRoy et al. 2002, Kimura et al. 1992 and Festuccia et al. 1997), or to inhibit these characteristics (Santibanez et al. 1996; Martinez et al. 1996).

Thus, in one context, the signals that originate from malignancies such as prostate that to induce bone proliferation can be extremely harmful. On the other hand, if their power could be harnessed for the positive, one might be able to treat a variety of bone loss situations. Thus, there remains a need to identify and utilize these factors.

### SUMMARY OF THE INVENTION

Thus, in accordance with the present invention, there is provided a method of inducing a stem cell to undergo osteogenic differentiation comprising (a) providing a stem cell; and (b) contacting said stem cell with at least one factor produced by a metastatic prostate cancer cell under conditions supporting growth of said stem cell, whereby said factor induces said stem cell to become an osteoprogenitor cell, a preosteoblast, or an osteoblast. The stem cell may be a mesenchymal stem cell, a hematopoietic stem cell, an embryonic stem cell, a tissue stem cell, or an embryonic carcinoma cell. The factor may be derived from a metastatic prostate cancer cell conditioned medium, which may be contacted with metastatic prostate cell condition medium.

The osteoblast may further produce tissue-like aggregates, and may form bone matrix, mineralize bone matrix, or produce bone. The stem cell may be located in a subject, such as one suffering from bone loss, bone damage, or bone destructive disease. The bone destructive disease may be osteoporosis, secondary osteoporosis, osteolytic bone cancer, Paget's Disease, endocrinological disorders, hypophsophatemia, hypocalcemia, renal osteodystrophy, hypoparathyroidism, hyperparathyroidism, or osteomalacia The method may further comprise contacting said stem cell with an additional osteogenic factor.

In another embodiment, there is provided a composition comprising medium condition by growth of a metastatic prostate cancer cell therein. In still another embodiment, there is provided a protein factor obtained from metastatic prostate cancer cell conditioned medium that induces stems cells to differentiate into osteoblasts. In still yet another embodiment, there is provided a method of obtaining a protein factor produced by a metastatic prostate cancer cell, wherein said factor induces stems cells to differentiate into osteoblasts, comprising (a) obtaining metastatic prostate cancer cell conditioned medium; and (b) separating protein and non-protein components of said medium.

In a further embodiment, there is provided a heat-labile protein factor obtained from a metastatic prostate cancer cell conditioned medium that induces ex vivo bone formation by osteoblasts. In still a further embodiment, there is provided a method of obtaining a protein factor produced by a metastatic prostate cancer cell, wherein said factor induces ex vivo bone formation by osteoblasts, comprising (a) obtaining metastatic prostate cancer cell conditioned medium; and (b) separating protein and non-protein components of said medium.

In yet another embodiment, there is provided a method of inducing an osteoblast to form bone comprising (a) providing an osteoblast; and (b) contacting said stem cell with at least one factor produced by a metastatic prostate cancer cell under conditions supporting growth of said osteoblast, whereby said osteoblast produces bone. The factor may be derived from a metastatic prostate cancer cell conditioned medium, and osteoblast may be contacted with the metastatic prostate cell condition medium. The osteoblast may or may not be located in a subject, such as one suffering from bone loss, bone damage, or bone destructive disease. The bone destructive disease may be osteoporosis, secondary osteoporosis, osteolytic bone cancer, Paget's Disease, endocrinological disorders, hypophsophatemia, hypocalcemia, renal osteodystrophy, hypoparathyroidism, hyperparathyroidism, or osteomalacia The method may further comprise contacting said osteoblast with an additional osteogenic factor.

In yet further embodiments, there is provided:

a method of separating an osteoblast inducing factor from metastatic prostate cancer cell conditioned medium comprising (a) obtaining metastatic prostate cancer cell conditioned medium; (b) fractionating components of metastatic prostate cancer cell conditioned medium; and (c) assaying for osteoblast inducing activity in fractions from (b), wherein a fraction possessing osteoblast inducing activity contain separated osteoblast inducing factor;

a method of separating a bone inducing factor from metastatic prostate cancer cell conditioned medium comprising (a) obtaining metastatic prostate cancer cell conditioned medium; (b) fractionating components of metastatic prostate cancer cell conditioned medium; and (c) assaying for bone inducing activity in fractions from (b), wherein a fraction possessing bone inducing activity contains separated bone inducing factor;

a method of identifying an osteoblast inducing factor from metastatic prostate cancer cell conditioned medium comprising (a) obtaining metastatic prostate cancer cell conditioned medium; (b) fractionating components of metastatic prostate cancer cell conditioned medium; (c) assaying for osteoblast inducing activity in fractions from (b); and (d) identifying the factor in the fraction of (c);

a method of identifying a bone inducing factor from metastatic prostate cancer cell conditioned medium comprising (a) obtaining metastatic prostate cancer cell conditioned medium; (b) fractionating components of metastatic prostate cancer cell conditioned medium; (c) assaying for bone inducing activity in fractions from (b); and (d) identifying the factor in the fraction of (c);

polyclonal antisera against metastatic prostate cancer cell conditioned medium;

a method of preparing an antibody population comprising (a) generating polyclonal antisera against metastatic prostate cancer cell conditioned medium; and (b) depleting said antisera of antibodies reactive with non-metastatic prostate cancer cell conditioned medium; and

a method of preparing a hybridoma cell comprising (a) generating a hybridoma cell population secreting antibodies against metastatic prostate cancer cell conditioned medium; and (b) depleting said hybridoma cell population of cells secreting antibodies reactive with non-metastatic prostate cancer cell conditioned medium.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Osteoinduction by Prostate Cancer (CaP) Cell Conditioned Media. Serum free conditioned media (CM) from CaP cell lines VcCaP and DuCaP were used to induce mesenchymal stem cells (MSC) to form tissue-like aggregates, and human bone (arrows). CM denatured by boiling or protease treatment lacks this activity. MSC; Mesenchymal stem cell.

FIG. 2. CaP-treated MSC Form Alizarin Red-positive Bone Matrix. MSC treated with CAP-CM form tissue-like aggregates containing bone matrix that statins positive for calcium phosphate with Alizarin red stain. Abbreviations as in FIG. 1.

FIG. 3. Raman Spectrographic Analysis of MSC Bone Formation. Mineralized bone matrix (shown as Alizarin red for illustration only) was further analyzed with Raman spectrometry. These spectrographs (upper and lower right boxes) clearly indicated the presence of hydroxyapatite, and collagen, confirming the formation of human bone.

FIG. 4. CaP-Secreted Growth Factors Fail to Induce Tissue-Like Aggregation, Osteoinduction or Bone Formation.

### DESCRIPTION OF ILLUSTRATIVE EMBODIMENTS

There is no curative treatment for lost bone mass, including various growth-promoting proteins and Vitamin D3. Likewise, there is no effective replacement or implant for non-union fractures or crush injuries of the bone. Currently, these latter types of injury utilize bovine, or human cadaver bone that is chemically treated to remove proteins, in order to prevent rejection. However, such bone implants, while mechanically important, are biologically “dead” (they do not contain bone-forming cells, growth factors, or other regulatory proteins) and do not greatly modulate the repair process.

Moreover, certain cancers, such as prostate, and breast are characterized by metastasis that induce localized bone proliferation. In the case of CaP, these cells clearly produce a signal that induces cells to produce bone tissue, whereas the others often form bone-eroding (osteolytic) lesions. Thus, the isolation of these factors not only provides a tool for inducing bone proliferation in damaged bone tissues, for halting erosion in osteolytic lesions, and their identification would permit intervention in osteogenic metastatic cancers.

The present invention concerns the observation that metastatic prostate cancer cells product a factor or factors that stimulate the formation of bone tissue. A distinguishing feature of such tissue is the formation of bone tissue-like aggregates or bone cell “spheroids.” The invention also provides for methods of implanting into subjects the cells leading to bone formation, the factor(s) inducing bone formation, or the ex vivo formed bone for the treatment of various bone diseases or bone fractures, breaks or other traumas. Also described are methods for screening prostate cancer cells for the factor or factors that lead to bone formation.

### I. Bone Formation

Under normal culture conditions, osteogenic cells are grown in the presence of varying amounts of serum, and remain adherent to the culture dish, essentially growing as a two dimensional, planar sheet of cells. The in vitro expansion of these cells requires their release from the plastic by trypsin treatment and re-culturing. After 4 to 6 weeks, the cells are placed in media containing serum and higher levels of calcium and phosphate. These cells reach confluent densities, and then “pile up” forming multi-layered cell structures referred to as bone nodules that mineralize their surrounding extracellular matrix.

In sharp contrast, osteogenic cells grown in serum-free conditions undergo a distinctly different developmental pattern resulting in the creation of a new composition of matter. This process requires the presence of TGF-β, or other osteogenic growth factors, added within the first 0 to 48 hours of culture. Under these conditions, the cells become plastic adherent for an additional 24 to 36 hour period; spontaneously release from the plastic surface of the tissue culture dish; form non-adherent variably sized three-dimensional spheroid-shaped cell aggregates, termed “bone cell spheroids.” The bone cells within the spheroids, due to their tissue-like three dimensional development, undergo cellular differentiation resulting in biochemical changes leading to rapid (3 to 7 day) mineralization of the spheroid, and the formation of crystalline bone-like structures (termed microspicules). The composition of matter described herein (of microspicule-containing cell spheroids) differs distinctly from normal bone cell cultures. First and foremost, this process results in spheroidal, three-dimensional, tissue-like development of bone cells. Second, these tissue-like aggregates are non-adherent, developing in suspension with the cells only adherent to each other and not the underlying culture dish plastic. Lastly, the tissue-like development of bone cells requires serum-free conditions and results in the ex vivo formation of 1 crystalline bone within the three dimensional tissue aggregate, a process not observed or described in any other culture system.

Stem cells can be isolated from a variety of sources: embryos, tissues, bone marrow, cell lines, or cancinomas. These cells are pluripotent, but can be cultured in the presence of factors that induce their commitment and differentiation into the bone cell lineage. Under normal culture conditions, stem cells are grown in the presence of varying amounts of serum. In sharp contrast, stem cell in the present invention are grown in serum-free conditions and undergo a distinctly different developmental pattern resulting in the creation of a new composition of matter. This process requires the presence of conditioned medium (CM) from CaP cells, alone or in the presence of osteogenic growth factors, added within the first 0 to 48 hours of culture. Once so cultured the undergo development into osteoprogenitor cells, preosteoblasts and/or osteoblasts. Subsequently, they rapidly (3-5 days) form tissue like aggregates as described above, but may take as long as 10-21 days, depending on the source of stem cell.

### II. Starting Cells

A. Stem Cells

In accordance with the present invention, stem cells are provided that may be treated with metastatic prostate cancer cell conditioned medium for the generation of osteogenic cells. Stem cells are generally defined as pluripotent cells that may be induced to differentiate into a particular cell type—in this case, bone forming cells. Various stem cells may be utilized in accordance with the present invention, and include mesenchymal stem cells, hematopoietic stem cells, embryonic stem cells, tissue stem cells, and embryonic carcinoma cells. These cells can be isolated by a variety of methods well know in the art, including immunological isolation, physico-chemical isolation, fluorescent activated cell sorting, limiting dilution culture, etc.

B. Metastatic Prostate Cancer Cells

The majority (70%) of human prostatic adenocarcinomas arise in the peripheral zone of the prostate. As the tumor develops, it begins to spread locally into the periprostatic fat, seminal vesicles and regional lymph nodes, specifically the hypogastric and obturator nodes. Once the tumor has breached the vascular bed, cancer cells will spread through the circulation (hematogenously) to distant sites where they become lodged in the capillary bed of permissive organs and form secondary tumors or metastases. The most common site of prostate cancer (CaP) metastasis is the axial skeleton, specifically the pelvis, femur and vertebral column, with lesser bony involvement in the ribs and skull. Distant visceral metastases are less common but include the liver, lung, and the dura of the brain. In general, the metastatic spread of cancer cells is a highly selective, non-random process. Only those tumor cells that can both respond to and manipulate the surrounding organ environment will ultimately form a metastatic lesion within the organ. With regards to CaP cells, bone marrow containing trabecular bone represents a permissive environment that can support metastatic CaP cell growth and survival. (Pazdur, 2002).

### III. Osteogenic Precursor Cells

The present invention provides for the generation, from stem cells, of a bone precusor (osteogenic) cells. As used herein, a bone precursor cell is any cell that is capable of differentiating or expanding into an osteoblast cell. The following section describes the characteristics of these cells. Osteogenic or precursor cells are derived from primary sources such as bone marrow or bone. In addition, cells can be derived from several different species, including cells of human, bovine, equine, canine, feline and murine origin.

A. Bone Precursor Cells (Osteoprogenitor cells)

Human bone precursor cells are characterized as small-sized cells that express low amounts of bone proteins (osteocalcin, osteonectin, and alkaline phosphatase) and have a low degree of internal complexity (Long et al., 1995). When stimulated to differentiate, these preosteoblast cells become osteoblast in their appearance, size, antigenic expression, and internal structure. Although these cells are normally present at very low frequencies in bone marrow, a process for isolating these cells has been described (Long et al., 1995). U.S. Pat. No. 5,972,703 further describes methods of isolating and using bone precursor cells, and is specifically incorporated herein by reference.

B. Preosteoblasts

Preosteoblasts are intermediate between osteoprogenitor cells and osteoblasts. The show increasing expression of bone phenotypic markers such as alkaline phosphatase (Kale et al., 2000). They have a more limited proliferative capacity, but nonetheless continue to divid and produce more preosteoblasts or osteoblasts.

C. Osteoblasts

Osteoblasts are the most mature cells of the bone cell lineage. They are large cells, possessing a eccentric nucleus, and produce of the extracellular proteins required for bone formation. They can be obtained from bone as populations of both preosteoblasts and osteoblasts as described in U.S. Ser. No. 09/753,043, which is specifically incorporated herein by reference.

### IV. Serum-Free Media

The present invention may be employed using serum-free media for growth of the metastaic prostate cancer cells, and the subsequent conversion of stem cells into bone-forming osteogenic or osteoprogenitor cells. The following section describes attributes and conditions for using serum-free media.

The use of serum-free culture for the manufacture of recombinant biopharmaceuticals from mammalian cells has been thoroughly reviewed (Barnes, 1987; Barnes and Sam, 1980; Broad et al., 1991; Jayme, 1991). The list of the main additives which are used as supplements for serum-free media is summarized by Barnes (1987) and Barnes and Sam (1980). Most commercially available serum-free media contain a carrier protein such as albumin. The presence of carrier protein might be required for protection of the cell viability.

An example of serum free culture medium can be found in U.S. Pat. No. 5,063,157, herein incorporated by reference. The media comprises, in addition to the base medium, transferrin, insulin, a peptone, a β-D-xylopyranose derivative, selenite and a biological polyamine. Another serum free cell growth medium for mammalian cells is disclosed in U.S. Pat. No. 4,443,546. This growth medium, in addition to the basic medium, contains seven ingredients. EPA 481 791 discloses a culture medium for CHO cells comprising water, an osmolality regulator, a buffer, an energy source, amino acids, an iron source, a growth factor and other optional components. The two media exemplified contain 19 and 17 components, respectively. Examples of potential additives to serum free media follow below.

A. Albumin

Albumin is preferably supplied in the form of bovine (BSA) or human serum albumin (HSA) in an effective amount for the growth of cells. Albumin provides a source of protein in the media. Albumin is thought to act as a carrier for trace elements and essential fatty acids. Preferably, the albumin used in the present formulations is free of pyrogens and viruses, and when necessary, is approved regulatory agencies for infusion into human patients. The HSA may be deionized using resin beads prior to use. The concentration of human serum albumin is 1-8 mg/ml, preferably 3-5 mg/ml, most preferably 4 mg/ml.

B. Soluble Carrier/Fatty Acid Complex

The albumin mentioned above could be substituted by a soluble carrier/essential fatty acid complex and a soluble carrier cholesterol complex which can effectively deliver the fatty acid and cholesterol to the cells. An example of such a complex is a cyclodextrin/linoleic acid, cholesterol and oleic acid complex. This is advantageous as it would allow for the replacement of the poorly characterized albumin with a well defined molecule. The use of cyclodextrin removes the need for the addition of human/animal serum albumin, thereby eliminating any trace undesired materials which the albumin would introduce into the media. The use of cyclodextrin simplifies the addition of specific lipophilic nutrients to a serum-free culture.

The lipophilic substances which can be complexed with cyclodextrin include unsaturated fatty acids such as linoleic acid, cholesterol and oleic acid. The linoleic acid, cholesterol and oleic acid are present in effective amounts and can be present in equal proportions such that the total amount is 0.001 to 100 μg/ml, preferably 0.1 to 10 μg/ml. The preparation of such complexes is known in the art and is described, for example, in U.S. Pat. No. 4,533,637, the entire contents of which is hereby incorporated by reference.

C. Iron Source

A source of iron in an effective amount and in a form that can be utilized by the cells can be added to the media. The iron can be supplied by saturating transferrin, its carrier molecule, in an effective amount. The transferrin may be derived from animal sera or recombinantly synthesized. It is understood that when transferrin is derived from an animal source, it is purified to remove other animal proteins, and thus is usually at least 99% pure. The transferrin concentration is usually between 80 and 500 μg/ml, preferably between 120 and 500 μg/ml, more preferably between 130 and 500 μg/ml, even more preferably between 275 and 400 μg/ml and most preferably 300 μg/ml. An iron salt, preferably a water soluble iron salt, such as iron chloride (e.g., FeCl₃.6H₂O) dissolved in an aqueous solution such as an organic acid solution (e.g., citric acid) is used to supply the iron to transferrin. One mole of iron chloride is usually used for every mole of citric acid. The concentration of iron chloride is 0.0008 to 8 μg/ml, preferably 0.08 to 0.8 μg/ml, most preferably 0.08 μg/ml.

D. Insulin Growth Factor

Insulin also may be added to the media of the present invention in an effective amount. The insulin concentration is between 0.25 and 2.5 U/ml, more preferably 0.4-2.1 U/ml, most preferably 0.48 U/ml. In the conversion of Units to mass, 27 U=1 mg. Therefore, incorporating the conversion, the insulin concentration is approximately between 9.26 ug/ml and 92.6 ug/ml, more preferably 14.8 ug/ml-77.8 ug/ml, most preferably 17.7 ug/ml. It is again understood that human insulin is more preferable than animal insulin. Highly purified recombinant insulin is most preferred. An insulin like growth factor such as insulin like growth factor 1 and insulin like growth factor 2 may be used in place of or in addition to insulin in an amount which provides substantially the same result as a corresponding amount of insulin. Thus, the term “insulin growth factor” includes both insulin and insulin like growth factors.

E. Additional Components

The addition of other lipids to the above essential reagents could enhance the proliferative potential of precursor cells. These components, however, are preferably not added unless they are necessary for a particular experiment or to grow a particular type of cell. Optionally, triglycerides and/or phospholipids may be included as additional sources of lipid. A preferable source of lipid contains a mixture of neutral triglycerides of predominantly unsaturated fatty acids such as linoleic, oleic, palmitic, linolenic, and stearic acid. Such a preparation may also contain phosphatidylethanolamine and phosphatidylcholine. Another source of lipid is a human plasma fraction precipitated by ethanol and preferably rendered virus free by pasteurization.

Other ingredients which can optionally be added to the media are cited in the following references: WO 95/06112, U.S. Pat. No. 4,533,637, U.S. Pat. No. 5,405,772. The entire contents of all of these references are incorporated by reference.

F. Undesired Components

When the media is to be used to grow cells for introduction into a human patient, the media preferably does not contain ingredients such as bovine serum albumin, mammalian serum, and/or any natural proteins of human or mammalian origin (as explained above). It is preferable that recombinant or synthetic proteins, if they are available and of high quality, are used. Most preferably, the amino acid sequences of the recombinant or synthetic proteins are identical to or highly homologous with those of humans. Thus, the most preferable serum-free media formulations herein contain no animal-derived proteins and do not have even a non-detectable presence of animal protein.

In the most ideal system, optional components which are not necessary are preferably not added to the medium. Such optional components are described in the prior art cited above and may be selected from the group consisting of meat extract, peptone, phosphatidylcholine, ethanolamine, anti-oxidants, deoxyribonucleosides, ribonucleosides, soy bean lecithin, corticosteroids, myoinositol, monothioglycerol, and bovine or other animal serum albumin.

V. Purification Methods

In various embodiments of the present invention, one will desired to fractionate molecules from cell conditioned medium. Any technique may prove useful, and can include chemical methods, such as phase partioning or precipitating (salting out), physical methods, such as chromatography, isoelectric focusing, centrifugation or electrophoresis, enzymatic methods (glycosylases, proteases, lipases, etc.), mass spectrometry, and even thermal (heating, freeze-thawing).

Any of a wide variety of chromatographic procedures may be employed according to the present invention. For example, thin layer chromatography, gas chromatography, high performance liquid chromatography, paper chromatography, affinity chromatography or supercritical flow chromatography may be used to effect separation of various chemical species.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatograph and thin-layer chromatograph (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillarity draws the solvent through the paper, dissolves the sample, and moves the components in the direction of flow. Paper chromatograms may be developed for either ascending or descending solvent flow. Two dimensional separations are permitted by changing the axis of migration 90° after the first run.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In Gas-Liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### VI. Antibodies

In one embodiment of the present invention, one will desire to prepare antibodies against the factor or factors contained in metastatic prostate cancer cell conditioned medium. An antibody can be a polyclonal or a monoclonal antibody. In one embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH), Multiple Antigenic Peptide (MAP), or bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition. The immunizing composition is administered in a manner effective to stimulate antibody producing cells. Rodents such as mice and rats are preferred animals, however, the use of rabbit, sheep, and frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×107 to 2×108 lymphocytes.

The antibody-producing B lymphocytes from the immunized animal are then fused with cells of an immortal myeloma cell, generally one of the same species as the animal that was inmmunized. Myeloma cell lines suited for use in hybridoma-producing fusion procedures preferably are non-antibody-producing, have high fusidn efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bu1; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10-6 to 1×10-8. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HHHHhhPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### VII. Diseases and Conditions Requiring Bone Repair

The following is a brief discussion of four human conditions to exemplify the variety of diseases and disorders that would benefit from the use of the factor or factors produced by metastatic prostate cancer cells. In addition to the following, several other conditions, such as, for example, vitamin D deficiency, exists.

The first example is the otherwise healthy individual who suffers a fracture. Often, clinical bone fracture is treated by casting to alleviate pain and allow natural repair mechanisms to repair the wound. There has been progress in the treatment of fracture in recent times, however, even without considering the various complications that may arise in treating fractured bones, any new procedures to increase bone healing in normal circumstances would be represent a great advance.

A second example which may benefit from new treatment methods is osteogenesis imperfecta (OI). OI encompasses various inherited connective tissue diseases that involve bone and soft connective tissue fragility in humans (Byers and Steiner, 1992; Prockop, 1990). About one child per 5,000-14,000 born is affected with OI and the disease is associated with significant morbidity throughout life. A certain number of deaths also occur, resulting from the high propensity for bone fracture and the deformation of abnormal bone after fracture repair (OI types II-IV; Bonadio and Goldstein, 1993). The relevant issue here is quality of life; clearly, the lives of affected individuals would be improved by the development of new therapies designed to stimulate and strengthen the fracture repair process.

OI type I is a mild disorder characterized by bone fracture without deformity, blue sclerae, normal or near normal stature, and autosomal dominant inheritance (Bonadio and Goldstein, 1993). Osteopenia is associated with an increased rate of lone bone fracture upon ambulation (the fracture frequency decreases dramatically at puberty and during young adult life, but increases once again in late middle age). Hearing loss, which often begins in the second or third decade, is a feature of this disease in about half the families and can progress despite the general decline in fracture frequency. Dentinogenesis imperfecta is observed in a subset of individuals.

In contrast, OI types II-VI represent a spectrum of more severe disorders associated with a shortened life-span. OI type II, the perinatal lethal form, is characterized by short stature, a soft calvarium, blue sclerae, fragile skin, a small chest, floppy appearing lower extremities (due to external rotation and abduction of the femurs), fragile tendons and ligaments, bone fracture with severe deformity, and death in the perinatal period due to respiratory insufficiency. Radiographic signs of bone weakness include compression of the femurs, bowing of the tibiae, broad and beaded ribs, and calvarial thinning.

OI type III is characterized by short stature, a triangular facies, severe scoliosis, and bone fracture with moderate deformity. Scoliosis can lead to emphysema and a shortened life-span due to respiratory insufficiency. OI type IV is characterized by normal sclerae, bone fracture with mild to moderate deformity, tooth defects, and a natural history that essentially is intermediate between OI type II and OI type I.

More than 150 OI mutations have been characterized since 1989 (reviewed in Byers and Steiner, 1992; Prockop, 1990). The vast majority occur in the COL1A1 and COL1A2 genes of type I collagen. Most cases of OI type I appear to result from heterozygous mutations in the COL1A1 gene that decrease collagen production but do not alter primary structure, i.e., heterozygous null mutations affect COL1A1 expression.

A third, important example is osteopenias/osteoporosis. The terms osteopenias and osteoporosis refers to a heterogeneous group of disorders characterized by decreased bone mass and fractures. Osteopenia is a bone mass that is one or more standard deveiations below the mean bone mass for a population; osteoporosis is deficed as 2.5 SD or lower. An estimated 20-25 million people are at increased risk for fracture because of site-specific bone loss. Risk factors for osteoporosis include increasing age, gender (more females), low bone mass, early menopause, race (Caucasians in general; asian and hispanic females), low calcium intake, reduced physical activity, genetic factors, environmental factors (including cigarette smoking and abuse of alcohol or caffeine), and deficiencies in neuromuscular control that create a propensity to fall.

More than a million fractures in the USA each year can be attributed to osteoporosis, and in 1986 alone the treatment of osteoporosis cost an estimated 7-10 billion health care dollars. Demographic trends (i.e., the gradually increasing age of the US population) suggest that these costs may increase to $62 billion by the year 2020. Clearly, osteoporosis is a significant health care problem.

Clinically, osteoporosis is segregated into type I and type II. Type I osteoporosis occurs predominantly in middle aged women and is associated with estrogen loss at the menopause, while osteoporosis type II is associated with advancing age. Much of the morbidity and mortality associated with osteoporosis results from immobilization of elderly patients following fracture.

Current therapies for osteoporosis patients focus on fracture prevention, not fracture repair. This remains an important consideration because of the literature, which clearly states that significant morbidity and mortality are associated with prolonged bed rest in the elderly, particularly those who have suffered hip fractures. Complications of bed rest include blood clots and pneumonia. These complications are recognized and measures are usually taken to avoid them, but these is hardly the best approach to therapy. thus, the osteoporotic patient population would benefit from new therapies designed to strengthen bone and speed up the fracture repair process, thus getting these people on their feet before the complications arise.

A fourth example is related to bone reconstruction and, specifically, the ability to reconstruct defects in bone tissue that result from traumatic injury; as a consequence of cancer or cancer surgery; as a result of a birth defect; or as a result of aging. There is a significant need for more frequent orthopedic implants, and cranial and facial bone are particular targets for this type of reconstructive need. The availability of new implant materials, e.g., titanium, has permitted the repair of relatively large defects. Titanium implants provide excellent temporary stability across bony defects. However, experience has shown that a lack of viable bone bridging the defect can result in exposure of the appliance, infection, structural instability and, ultimately, failure to repair the defect.

Autologous bone grafts are another possibility, but they have several demonstrated disadvantages in that they must be harvested from a donor site such as iliac crest or rib, they usually provide insufficient bone to completely fill the defect, and the bone that does form is sometimes prone to infection and resorption. Partially purified xenogeneic preparations are not practical for clinical use because microgram quantities are purified from kilograms of bovine bone, making large scale commercial production both costly and impractical. Allografts and demineralized bone preparations are therefore often employed.

Microsurgical transfers of free bone grafts with attached soft tissue and blood vessels can close bony defects with an immediate source of blood supply to the graft. However, these techniques are time consuming, have been shown to produce a great deal of morbidity, and can only be used by specially trained individuals. Furthermore, the bone implant is often limited in quantity and is not readily contoured. In the mandible, for example, the majority of patients cannot wear dental appliances using presently accepted techniques (even after continuity is established), and thus gain little improvement in the ability to masticate. Toriumi et al. (1991) have written the “Reconstructive surgeons should have at their disposal a bone substitute that would be reliable, biocompatible, easy to use, and long lasting and that would restore mandibular continuity with little associated morbidity.”

In connection with bone reconstruction, specific problem areas for improvement are those concerned with treating large defects, such as created by trauma, birth defects, or particularly, following tumor resection; and also the area of artificial joints. The success of orthopaedic implants, interfaces and artificial joints could conceivably be improved if the surface of the implant, or a functional part of an implant, were to be coated with a bone stimulatory agent. The surface of implants could be coated with one or more appropriate materials in order to promote a more effective interaction with the biological site surrounding the implant and, ideally, to promote tissue repair.

### VIII. Polymers for Implanting of Bone Cells

Over the last decade there has been a tremendous increase in applications for polymeric materials. These materials are well suited to implantation as they can serve as a temporary scaffold to be replaced by host tissue, degrade by hydrolysis to non-toxic products, and be excreted, as described by Kulkarni et al. (1971) and Hollinger and Battistone (1986).

Either natural or synthetic polymers can be used to form the matrix, although synthetic polymers are preferred for reproducibility and controlled release kinetics. Synthetic polymers that can be used include bioerodible polymers such as poly(lactide) (PLA), poly(glycolic acid) (PGA), poly(lactide-co-glycolide) (PLGA), and other poly(alpha-hydroxy acids), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, and nylon. Although non-degradable materials can be used to form the matrix or a portion of the matrix, they are not preferred. Examples of natural polymers include proteins such as albumin, fibrin or fibrinogen, collagen, synthetic polyamino acids, and prolamines, and polysaccharides such as alginate, heparin, and other naturally occurring biodegradable polymers of sugar units.

Four polymers widely used in medical applications are poly(paradioxanone) (PDS), poly(lactic acid) (PLA), poly(glycolic acid) (PGA), and PLAGA copolymers. Copolymerization enables modulation of the degradation time of the material. By changing the ratios of crystalline to amorphous polymers during polymerization, properties of the resulting material can be altered to suit the needs of the application. These polymers, including poly(lactide-co-glycolic) acid (PLGA), have been used as polymer composites for bone replacement as reported by Elgendy et al. (1993). Substituted polyphosphazenes have been shown to support osteogenic cell growth, as reported by Laurencin et al. (1993). Poly(organophosphazenes) are high molecular weight polymers containing a backbone of alternating phosphorus and nitrogen atoms. There are a wide variety of polyphosphazenes, each derived from the same precursor polymer, poly(dichlorophosphazene). The chlorine-substituted species can be modified by replacement of the chlorine atoms by different organic nucleophiles such as o-methylphenoxide along with amino acids. The physical and chemical properties of the polymer can be altered by adding various ratios of hydrolytic sensitive side chains such as ethyl glycinate, as described by Wade et al. (1978) and Allcock and Fuller (1981). This will affect the degradation of the polymer as an implantable and biodegradable material as well as vary the support of osteogenic cells for bone and tissue implants, as shown by Laruencin et al. (1993).

PLA, PGA and PLA/PGA copolymers are particularly useful for forming the biodegradable matrices. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. Nos. 1,995,970; 2,703,316; 2,758,987; 2,951,828; 2,676,945; 2,683,136; and 3,531,561. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in Cyanamid Research Develops World's First Synthetic Absorbable Suture,” Chemistry and Industry, 905 (1970).

The erosion of the matrix is related to the molecular weights of PLA, PGA or PLA/PGA. The higher molecular weights, weight average molecular weights of 90,000 or higher, result in polymer matrices which retain their structural integrity for longer periods of time; while lower molecular weights, weight average molecular weights of 30,000 or less, result in both slower release and shorter matrix lives. Poly(lactide-co-glycolide) (50:50), degrades in about six weeks following implantation.

All polymers for use in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

These polymers are particularly useful in forming fibrous or sponge type matrices for implantation. Polymers can also be used to form hydrogels in which the cells are suspended and then implanted.

A. Other Matrix Materials

Another class of materials for making the matrix is hydroxyapatite, or a similar ceramic formed of tricalcium phosphate (TCP) or calcium phosphate (CaPO₄). Calcium hydroxyapatites occur naturally as geological deposits and in normal biological tissues, principally bone, cartilage, enamel, dentin, and cementum of vertebrates and in many sites of pathological calcifications such as blood vessels and skin. Synthetic calcium hydroxyapatite is formed in the laboratory either as pure Ca₁₀ (PO₄)₆ (OH)₂ or hydroxyapatite that is impure, containing other ions such as carbonate, fluoride, chloride for example, or crystals deficient in calcium or crystals in which calcium is partly or completely replaced by other ions such as barium, strontium and lead. Essentially none of the geological and biological apatites are “pure” hydroxyapatite since they contain a variety of other ions and cations and may have different ratios of calcium to phosphorous than the pure synthetic apatites.

In general, the crystals of pure synthetic apatites, geological apatites and many impure synthetically produced apatites are larger and more crystalline than the biological crystals of bone, dentin, cementum and cartilage. The crystals of bone, dentin and cementum are very small, irregularly shaped, very thin plates whose rough average dimensions are approximately 10 to 50 angstroms in thickness, 30 to 150 angstroms in width, and 200 to 600 angstroms in length. The synthetic materials are highly diverse, as reported in the literature. For example, the characterization of four commercial apatites was reported by Pinholt et al. (1992); Marden et al. (1990) reports on a protein, biodegradable material; Pinholt et al. (1991) reports on the use of a bovine bone material called Bio-Oss™; Friedman et al. (1991) and Costantino et al. (1991) report on a hydroxyapatite cement; Roesgen (1990) reports on the use of calcium phosphate ceramics in combination with autogenic bone; Ono et al. (1990) reports on the use of apatite-wollastonite containing glass ceramic granules, hydroxyapatite granules, and alumina granules; Passuti et al. (1989) reports on macroporous calcium phosphate ceramic performance; Harada (1989) reports on the use of a mixture of hydroxyapatite particles and tricalcium phosphate powder for bone implantation; Ohgushi et al. (1989) reports on the use of porous calcium phosphate ceramics alone and in combination with bone marrow cells; Pochon et al. (1986) reports on the use of beta-tricalcium phosphate for implantation; and Glowacki et al. (1985), reports on the use of demineralized bone implants.

As used herein, all of these materials are generally referred to as “hydroxyapatite.” In the preferred form, the hydroxyapatite is particles having a diameter between approximately ten and 100μ in diameter, most preferably about 50μ in diameter.

Calcium phosphate ceramics can be used as implants in the repair of bone defects because these materials are non-toxic, non-immunogenic, and are composed of calcium and phosphate ions, the main constituents of bone (Frame, 1987; Parsons et al., 1988). Both tricalcium phosphate (TCP) Ca₃(PO₄)₂ and hydroxyapatite (HA) Ca₁₀ (PO₄)₆ (OH₂) have been widely used. Calcium phosphate implants are osteoinductive, and have the apparent ability to become directly bonded to bone. As a result, a strong bone-implant interface is created.

Calcium phosphate ceramics have a degree of bioresorbability which is governed by their chemistry and material structure. High density HA and TCP implants exhibit little resorption, while porous ones are more easily broken down by dissolution in body fluids and resorbed by phagocytosis. However, TCP degrades more quickly than HA structures of the same porosity in vitro. HA is relatively insoluble in aqueous environments. However, the mechanical properties of calcium phosphate ceramics make them ill-suited to serve as a structural element under load bearing circumstances. Ceramics are not preferred since they are brittle and have low resistance to impact loading.

B. Polymers for Forming Hydrogels

Polymers that can form ionic hydrogels which are malleable can also be used to support the cells. Injecting a suspension of cells in a polymer solution may be performed to improve the reproducibility of cell seeding throughout a device, to protect the cells from shear forces or pressure induced necrosis, or to aid in defining the spatial location of cell delivery. The injectable polymer may also be utilized to deliver ells and promote the formation of new tissue without the use of any other matrix. In a preferred embodiment, the hydrogel is produced by cross-linking the ionic salt of a polymer with ions, whose strength increases with either increasing concentrations of ions or polymer. The polymer solution is mixed with the cells to be implanted to form a suspension, which is then injected directly into a patient prior to polymerization of the suspension. The suspension subsequently polymerizes over a short period of time due to the presence in vivo of physiological concentrations of ions such as calcium in the case where the polymer is a polysaccharide such as alginate.

A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazenes, and polyacrylates such as hydroxyethyl methacrylate (HEMA), which are crosslinked ionically, or block copolymers such as Pluronics™ or Tetronics™, polyethylene oxide-polypropylene glycol block copolymers which are crosslinked by temperature or pH, respectively. Other materials include proteins such as fibrinogin, collagen, polymers such as polyvinylpyrrolidone, hyaluronic acid and collagen.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Alginate can be ionically cross-linked with divalent cations, in water, at room temperature, to form a hydrogel matrix. Due to these mild conditions, alginate has been the most commonly used polymer for hybridoma cell encapsulation, as described, for example, in U.S. Pat. No. 4,352,883. Described therein is an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolyrically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol and glucosyl.

The water soluble polymer with charged side groups is crosslinked by reacting the polymer with an aqueous solution containing multivalent ions of the opposite charge, either multivalent cations if the polymer has acidic side groups or multivalent anions if the polymer has basic side groups. The preferred cations for cross-linking of the polymers with acidic side groups to form a hydrogel are divalent and trivalent cations such as copper, calcium, aluminum, magnesium, strontium, barium, and tin, although di-, tri- or tetra-functional organic cations such as alkylammonium salts can also be used. Aqueous solutions of the salts of these cations are added to the polymers to form soft, highly swollen hydrogels and membranes. The higher the concentration of cation, or the higher the valence, the greater the degree of cross-linking of the polymer. Concentrations from as low as 0.005M have been demonstrated to cross-link the polymer. Higher concentrations are limited by the solubility of the salt. The preferred anions for cross-linking of the polymers to form a hydrogel are divalent and trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine), examples of synthetic polyamines are polyethyleneimine, poly(vinylamine), and poly(allyl amine). There are also natural polycations such as the polysaccharide, chitosan. Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO₃ H groups such as sulfonated polystyrene, and polystyrene with carboxylic acid groups.

### IX. Screening Assays

In still further embodiments, the present invention provides methods for separating, purifying and identifying factors that induce bone formation. Using the separative techniques described above, conditioned medium may be fractionated and the subsequent fractions tested for activity, such as osteoblast-inducing activity or bone-inducing activity. Osteoblasts are identified by their morphology, expression of bone proteins, and ability to mineralize matrix and/or produce bone. Bone formation can be identified by the von Koassa or Alzarin Red stains, FTIR or Raman spectrometric analysis.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The inventors utilized primary human osteoblasts and CaP cell lines to explore the cellular basis of the osteoblastic CaP lesions. Primary human preosteoblasts and osteoblasts and CaP cell lines were used to explore the cellular basis of the osteoblastic CaP lesions. The data show that CaP cell lines (LNCaP, C42B, DuCaP and VCaP) co-cultured with human trabecular bone derived osteoblasts failed to stimulate either osteoblast proliferation or differentiation (as monitored by alkaline phosphatase enzymatic activity, data not shown). These data suggested that cellular recruitment to the bone cell lineage might play a role in CaP mediated osteoenesis.

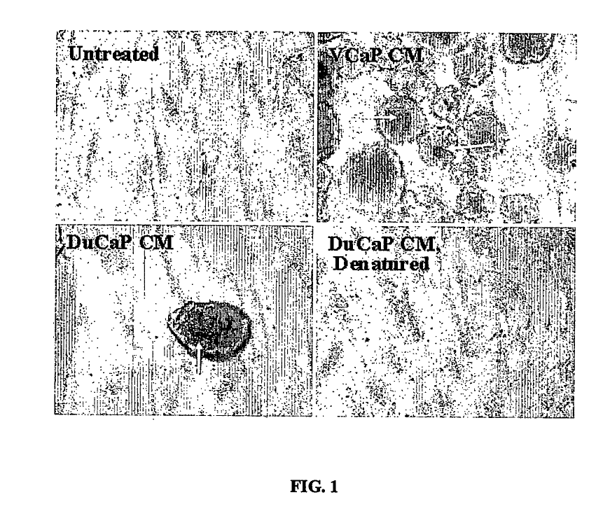

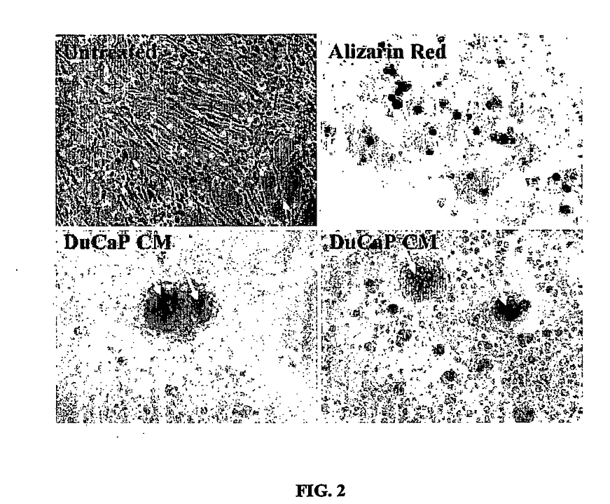

Mesenchymal stem cells (MSC) are pluripotent bone marrow cells that have osteogenic potential. The inventors observed that conditioned medium (CM) produced by metastatic human CaP cells (DuCaP and VCaP) induced MSC commitment and differentiation into osteoblasts (FIGS. 1 and 2). This activity is inhibited by either boiling or protease activity (FIG. 1 bottom right panel), indicating its protein nature. Importantly, CaP-CM induces osteoblast cellular condensation into tissue-like aggregates (spheroidal cell structures in FIG. 1 and 2). In turn, these tissue-like aggregates secreted and mineralized a bone matrix that was both von Kossa and Alizarin red positive (FIG. 2).

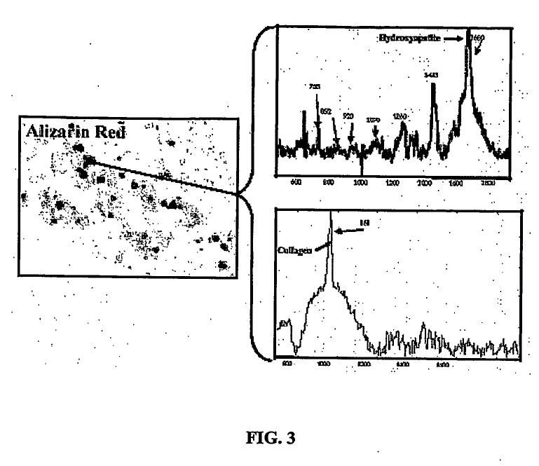

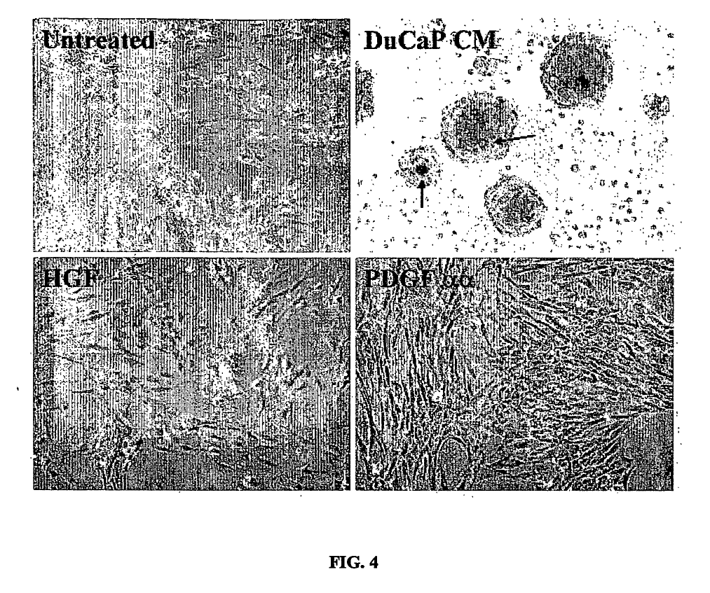

Raman spectrographic analysis confirmed the presence of both hydroxyapatite and collagen type I indicative of bone formation (FIG. 3). DNA microarray analysis detected that these CaP cells secrete a number of known growth factors (Table 1). How-ever, none of these could substitute for the CaP condition media activity indicating the CM contains a unique bone cell regulator (FIG. 4). The inventors conclude that CaP cells elaborate soluble protein(s) that recruit primitive cells to the osteoblast lineage and promote increased bone formation.

Conditioned medium from metastatic prostate cancer (CaP) cells is made in the following manner. First 4×10⁶ CaP cells (VCAP or DuCaP) are plated in 12 ml of culture medium (10% FBS DMEM medium) to 100 mm tissue culture dishes. Cells are then given 24 hours to adhere to the plastic surface. The following day, the medium is removed and the cells are washed 3× with 10 ml/dish PBS. Washed cells are then incubated 72 hours in 10 ml/dish serum-free McCoy's medium supplemented with 1% (v/v) ITS+. Medium collected after 72 hours is filtered through 0.2 mm filter and used directly in osteoinduction experiments, typically as a 1:2 dilution.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XI. REFERENCES

The following references, to the extent that they provide exemplary procedural or other details supplementary to those set forth herein, are specifically incorporated herein by reference.

U.S. Pat. No. 1,995,970

U.S. Pat. No. 2,676,945

U.S. Pat. No. 2,683,136

U.S. Pat. No. 2,703,316

U.S. Pat. No. 2,758,987

U.S. Pat. No. 2,951,828

U.S. Pat. No. 3,531,561

U.S. Pat. No. 4,196,265

U.S. Pat. No. 4,352,883

U.S. Pat. No. 4,443,546

U.S. Pat. No. 4,533,637

U.S. Pat. No. 5,063,157

U.S. Pat. No. 5,405,772

U.S. Pat. No. 5,972,703

U.S. Ser. No. 09/753,043

Cynamid Research Develops World's First Synthetic Absorbable Suture, Chemistry and Industry, 905, 1970.

Allcock and Fuller, J. Am. Chem. Soc., 103:2250-2256, 1981.

Barnes and Sam, “Serum-free cell culture: A unifying approach,” Cell, 22:649-655, 1980.

Barnes, “Serum-free animal cell culture,” Bio. Techniques, 5: 534-542, 1987.

Bonadio and Goldstein, In: Molecular and Cellular Biology of Bone, Noda (Ed.), Academic Press, Inc., San Diego, 169-189, 1993.

Broad et al., Cytotechnology 5:47-55, 1991.

Byers and Steiner, Annu. Rev. Med., 43:269-289, 1992.

Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.

Costantino et al., Arch. Otolaryngol. Head Neck Surg., 117(4):379-384, 1991.

Elgendy et al., Biomaterials, 14:263-269, 1993.

EP 481 791

Festuccia et al., Oncology Res., 9(8):419-31, 1997.

Frame, Int. J. Oral Maxillofac. Surg., 16(6):642-655, 1987.

Friedman et al., Arch. Otolaryngol. Head Neck Surg., 117(4), 386-389, 1991.

Gefter et al., Somatic Cell Genet., 3:231-236, 1977.

Glowacki et al., Clin. Plast. Surg., 12(2), 233-241, 1985.

Goding, In: Monoclonal Antibodies: Principles and Practice, 2d ed., Academic Press, FL., 60-66, and 71-74, 1986.

Harada, Shikwa-Gakuho, 89(2):263-297, 1989.

Harlow and Lane, Antibodies “A Laboratory Manual, Cold Spring Harbor Laboratory, 1988.

Hollinger and Battistone, Clinical Orthop. Rel. Res., 207:290-305, 1986.

Jayme, Cytotechnology 5(1):15-30, 1991.

Kale et al., Nat. Biotech., 18:954-958, 2000.

Kimura et al., Urologia Intenzationalis., 48(1):25-30, 1992.

Kohler and Milstein, Eur. J. Immunol., 6:511-519, 1976.

Kohler and Milstein, Nature, 256:495-497, 1975.

Kulkarni et al., J. Biom. Mater. Res., 5:169-81, 1971.

Laurencin et al., J. Biom. Mater. Res., 27(7):963-973, 1993.

LeRoy et al., Prostate, 50(2):104-111, 2002

Long, J. Clin. Invest., 95:881-887, 1995.

Marden et al., J. Craniofac. Surg., 1(3):154-160, 1990.

Martinez et al., J. Cellular Biochem., 61(1):18-25, 1996.

Ohgushi et al., Acta Orthop. Scand., 60(3):334-339, 1989.

Ono et al., Biomaterials, 11(4):265-271, 1990.

Parsons et al., Ann. NY Acad. Sci., 523:190-207, 1988.

Passuti et al., Clin. Orthop., 248:169-176, 1989.

Pazdur, In: Cancer Management: A Multidisciplinary Approach, PPR Inc., NY, 2002.

Pinholt et al., J. Oral Maxillofac. Surg., 50(8):859-867, 1992.

Pinholt et al., Scand. J. Dent. Res., 99(2):154-161, 1991.

Pochon et al., Z-Kinderchir., 41(3):171-173, 1986.

Prockop, J. Biol. Chem., 265:15349-15352, 1990.

Roesgen, Unfallchirurgle, 16(5):258-265, 1990.

Santibanez et al. Brit. J. Cancer, 74(3):418-22, 1996

Toriumi et al., Arch. Otolaryngol Head Neck Surg., 117:1101-1112, 1991.

Wade et al., In: Organomet. Polym., Carraher et al. (Eds.), Academic Press, NY, 283-288, 1978.

WO 95/06112.

1. A method of inducing a stem cell to undergo osteogenic differentiation comprising:
(a) providing a stem cell; and
(b) contacting said stem cell with at least one factor produced by a metastatic prostate cancer cell under conditions supporting growth of said stem cell,
whereby said factor induces said stem cell to become an osteoprogenitor cell, a preosteoblast, or an osteoblast.

2. The method of claim 1, wherein said stem cell is a mesenchymal stem cell, a hematopoietic stem cell, an embryonic stem cell, a tissue stem cell, or an embryonic carcinoma cell.

3. The method of claim 1, wherein said factor is derived from a metastatic prostate cancer cell conditioned medium.

4. The method of claim 3, wherein said stem cell is contacted with metastatic prostate cell condition medium.

5. The method of claim 1, wherein said osteoblast further produces tissue-like aggregates.

6. The method of claim 5, wherein said tissue-like aggregates form bone matrix.

7. The method of claim 1, wherein said stem cell is located in a subject.

8. The method of claim 7, wherein said subject suffers from bone loss, bone damage, or bone destructive disease.

9. The method of claim 8, wherein said bone destructive disease is osteoporosis, secondary osteoporosis, osteolytic bone cancer, Paget's Disease, endocrinological disorders, hypophsophatemia, hypocalcemia, renal osteodystrophy, hypoparathyroidism, hyperparathyroidism, or osteomalacia.

10. The method of claim 1, further comprising contacting said stem cell with an additional osteogenic factor.

11. A composition comprising medium condition by growth of a metastatic prostate cancer cell therein.

12. A protein factor obtained from metastatic prostate cancer cell conditioned medium that induces stems cells to differentiate into osteoblasts.

13. A method of obtaining a protein factor produced by a metastatic prostate cancer cell, wherein said factor induces stems cells to differentiate into osteoblasts, comprising:
(a) obtaining metastatic prostate cancer cell conditioned medium; and
(b) separating protein and non-protein components of said medium.

14. A heat-labile protein factor obtained from a metastatic prostate cancer cell conditioned medium that induces ex vivo bone formation by osteoblasts.

15. A method of obtaining a protein factor produced by a metastatic prostate cancer cell, wherein said factor induces ex vivo bone formation by osteoblasts, comprising:
(a) obtaining metastatic prostate cancer cell conditioned medium; and
(b) separating protein and non-protein components of said medium.

16. A method of inducing an osteoblast to form bone comprising:
(a) providing an osteoblast; and
(b) contacting said stem cell with at least one factor produced by a metastatic prostate cancer cell under conditions supporting growth of said osteoblast, whereby said osteoblast produces bone.

17. The method of claim 1, wherein said factor is derived from a metastatic prostate cancer cell conditioned medium.

18. The method of claim 17, wherein said osteoblast is contacted with metastatic prostate cell condition medium.

19. The method of claim 17, wherein said osteoblast is located in a subject.

20. The method of claim 17, wherein said osteoblast is not located in a subject.

21. The method of claim 20, wherein said subject suffers from bone loss, bone damage, or bone destructive disease.

22. The method of claim 21, wherein said bone destructive disease is osteoporosis, secondary osteoporosis, osteolytic bone cancer, Paget's Disease, endocrinological disorders, hypophsophatemia, hypocalcemia, renal osteodystrophy, hypoparathyroidism, hyperparathyroidism, or osteomalacia.

23. The method of claim 16, further comprising contacting said osteoblast with an additional osteogenic factor.

24-30. (canceled)
